(19) United States
(12) Patent Application Publication
(10) Pub. No.: US 2021/0338918 A1
(43) Pub. Date: Nov. 4, 2021

(54) ROTATIONALLY ACTUATED NEGATIVE-PRESSURE WOUND THERAPY DEVICE

(71) Applicant: KCI Licensing, Inc., San Antonio, TX (US)

(72) Inventors: Benjamin Andrew PRATT, Poole (GB); Christopher Brian LOCKE, Bournemouth (GB)

(21) Appl. No.: 17/285,381

(22) PCT Filed: Sep. 27, 2019
(86) PCT No.: PCT/US2019/053541

§ 371 Date: Apr. 14, 2021

Related U.S. Application Data
(60) Provisional application No. 62/746,219, filed on Oct. 16, 2018.

(51) Int. Cl. A61M 1/00; F04B 45/04

(52) U.S. Cl. CPC A61M 1/82; A61M 1/90; F04B 45/04; A61M 2205/071; A61M 2205/8281; A61M 2205/103; A61M 2205/584

(57) ABSTRACT

A negative-pressure source is described. The negative-pressure includes a first housing and a second housing configured to be coupled to the first housing to form a cavity. The first housing and the second housing are rotatable on a common axis relative to each other. A membrane is disposed in the cavity and sealed to the second housing to form a first chamber and a second chamber. A motor is disposed in the first chamber, and a clockwork is coupled to the motor and configured to be driven by the motor. A threaded body is disposed in the first chamber and configured to be driven by the motor. The threaded body is configured to rotate on the common axis and to displace the membrane in response to rotation on the common axis.

### TECHNICAL FIELD

The invention set forth in the appended claims relates generally to tissue treatment systems and more particularly, but without limitation, to a device for generating a negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for treating a tissue site in a negative-pressure therapy environment are set forth in the appended claims. For example, a rotationally actuated negative-pressure source is provided that provides linear negative pressure and is operable by users having decreased dexterity and strength. The negative-pressure source can have an increased static capacity over other manually actuated negative-pressure sources while maintaining a size that is unobtrusive and easily charged and re-charged. Furthermore, the negative-pressure source can provide assistance or reminders to patients and care givers that dressing changes or therapy termination are imminent. Illustrative embodiments are also provided to enable a person skilled in the art to make and use the claimed subject matter.

In some embodiments, a negative-pressure source is described. The negative-pressure source can include a first housing and a second housing. The second housing can be configured to be coupled to the first housing to form a cavity. The first housing and the second housing can be rotatable on a common axis relative to each other. A membrane can be disposed in the cavity and sealed to the second housing to form a first chamber and a second chamber. A motor can be disposed in the first chamber, and a clockwork can be coupled to the motor and configured to be driven by the motor. A threaded body can be disposed in the first chamber and configured to be driven by the motor. The threaded body can be configured to rotate on the common axis and to displace the membrane in response to rotation on the common axis.

In other embodiments, rotation of the first housing relative to the second housing energizes the motor. The rotation of the threaded body can be configured to displace the membrane axially, generating a negative pressure in the second chamber. The clockwork can be configured to drive a charge status indicator, and the clockwork can be configured to drive a life status indicator.

More generally, another negative-pressure source is described. The negative-pressure source can include a bottom housing having a cylindrical shape, a first end, and a second end, and a cavity depending from the first end toward the second end. A diaphragm can be disposed in the cavity. The diaphragm can have a peripheral portion configured to seal to the bottom housing to form a pump chamber. The negative-pressure source can include a screw having a first end and a second end, the second end being coupled to the diaphragm. A carriage can be coupled to the first end of the screw, the carriage being operable to drive the screw. A motor can be disposed in the carriage, and a top housing can be coupled to the motor. The top housing can be configured to rotate relative to the bottom housing to energize the motor. A clockwork can be coupled to the top housing and configured to be driven by the motor.

In some embodiments, the negative-pressure source can include an absorbent disposed in the pump chamber. An inlet can be coupled to the bottom housing and fluidly coupled to the pump chamber, and a one-way valve can be coupled to the inlet. The one-way valve can be configured to permit fluid communication into the pump chamber and prevent fluid communication out of the pump chamber. In some embodiments, a threaded body can be disposed in the pump chamber and coupled to the diaphragm, the threaded body configured to be threaded to the second end of the screw. In some embodiments, a seal can be configured to be threaded to the second end of the screw and coupled to an opposite side of the diaphragm from the threaded body. At least one of the threaded body and the seal can have a peripheral portion configured to engage a wall of the bottom housing to center the screw in the pump chamber.

In some embodiments, the carriage can include a cylindrical body having a first end and a second end and a carriage cavity depending into the cylindrical body from the first end toward the second end. The screw can be coupled to the carriage opposite the carriage cavity.

In some embodiments, the negative-pressure source can include a lockout coupled to the top housing. The lockout can be configured to permit rotation of the top housing relative to the bottom housing in a single direction. In some embodiments, the clockwork is configured to drive a charge status indicator. In other embodiments, the clockwork is configured to drive a life status indicator.

Alternatively, other example embodiments may describe a method for providing negative-pressure therapy. A dressing can be disposed adjacent a tissue site. A negative-pressure source can be provided. The negative pressure source can include a bottom housing having a cylindrical shape, a first end, a second end, and a cavity depending from the first end toward the second end. A diaphragm can be disposed in the cavity. The diaphragm can have a peripheral portion configured to seal to the bottom housing to form a pump chamber. The negative-pressure source can include a screw having a first end and a second end, the second end being coupled to the diaphragm. A carriage can be coupled to the first end of the screw, the carriage being operable to drive the screw. A motor can be disposed in the carriage, and a top housing can be coupled to the motor. The top housing can be configured to rotate relative to the bottom housing to energize the motor. A clockwork can be coupled to the top housing and configured to be driven by the motor. The negative-pressure source can be fluidly coupled to the dressing. The top housing can be rotated relative to the bottom housing to displace the diaphragm, and fluid can be drawn from the dressing into the pump chamber.

In some embodiments, the rotation of the top housing relative to the bottom housing drives a charge status indicator. In some embodiments, rotation of the top housing relative to the bottom housing drives a life status indicator. In some embodiments, rotating the top housing relative to the bottom housing can energize the motor, rotate the carriage; and rotate the screw. In some embodiments, rotating the screw can move a seal threaded to the screw axially, the seal being disposed in the pump chamber and coupled to the diaphragm. Axial motion of the seal can draw the diaphragm toward the carriage and expand the pump chamber.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a perspective view illustrating additional details of the negative-pressure source that may be associated with the therapy system of FIG. 1;

FIG. 3 is a plan view illustrating additional details of the negative-pressure source of FIG. 2 in a first position;

FIG. 4 is a plan view illustrating additional details of the negative-pressure source of FIG. 2 in a second position;

FIG. 5 is a plan view illustrating additional details of another negative-pressure source of FIG. 2 in a first position;

FIG. 6 is a plan view illustrating additional details of the negative-pressure source of FIG. 5 in a second position;

FIG. 7 is a plan view illustrating additional details of an indicator of a negative-pressure source that may be associated with the therapy system of FIG. 1;

FIG. 8 is a sectional view of FIG. 7 taken along line 8-8 illustrating additional details of the indicator;

FIG. 9 is a plan view illustrating additional details of the indicator of FIG. 7;

FIG. 10 is a plan view illustrating additional details of another negative-pressure source that may be associated with the therapy system of FIG. 1;

FIG. 11 is a perspective view illustrating additional details that may be associated with the negative-pressure source of FIG. 10;

FIG. 12 is a front view illustrating additional details of an indicator that may be associated with the therapy system of FIG. 1;

FIG. 13 is an assembly view illustrating additional details of another negative-pressure source that may be associated with the therapy system of FIG. 1;

FIG. 14 is a perspective view illustrating additional details of another indicator that may be associated with the therapy system of FIG. 1;

FIG. 15 is a perspective view illustrating additional details that may be associated with the indicator of FIG. 14;

FIG. 16 is a perspective view illustrating additional details of another negative-pressure source that may be associated with the therapy system of FIG. 1;

FIG. 17 is an assembly view illustrating additional details of the negative-pressure source of FIG. 16; and

FIG. 18 is a sectional view illustrating additional details of a regulator that may be associated with the therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

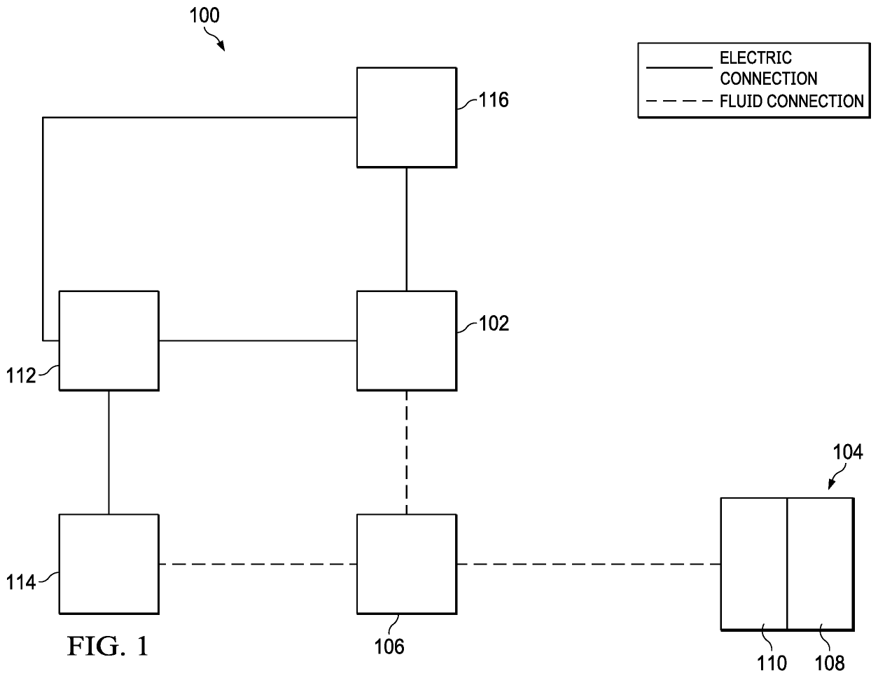

FIG. 1 is a simplified functional block diagram of an example embodiment of a therapy system 100 that can provide negative-pressure therapy to a tissue site in accordance with this specification. The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Tex.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112 and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 114 and the second sensor 116, are generally known in the art as an apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid across the tissue interface 108 under pressure. For example, a manifold may be adapted to receive negative pressure from a source and distribute negative pressure through multiple apertures across the tissue interface 108, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. For example, the tensile strength of foam may be increased for instillation of topical treatment solutions. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface 108 may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Tex.

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours measured using an upright cup technique according to ASTM E96/E96M Upright Cup Method at 58° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 5M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 110 may comprise INSPIRE 2301 having an MVTR (upright cup technique) of 2600 g/m²/24 hours and a thickness of about 50 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 108 in the sealed therapeutic environment can induce macro-strain and micro-strain in the tissue site. Negative pressure can also remove exudate and other fluid from a tissue site, which can be collected in the container 106.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108. In other embodiments, the negative-pressure source 102 may have a therapy pressure set at a manufacturing facility. Different negative-pressure sources 102 may be set with different therapy pressures. The controller 112 can be a mechanical pressure regulator that receives one or more inputs, for example, a pressure at a tissue site, and a pressure at the negative-pressure source 102. The controller 112 can regulate the fluid communication between the negative-pressure source 102 and the tissue site to provide the therapy pressure to the dressing 104.

Some therapy units can be portable and operate independently of an electrical connection or an electrical storage device, such as a battery. A portable therapy unit may use a mechanical assembly to develop a negative-pressure without an electrically-powered pump. In some cases, the mechanical assembly may be a consumable component capable of being operated only once. For example, the mechanical assembly of a portable therapy unit may be operated a single time by a user to charge the therapy unit. As used herein, charging a therapy unit can refer to the operation of the therapy unit to generate a negative pressure in an associated dressing, such as the dressing 104. If the mechanical assembly is consumable, the therapy unit may only be charged a single time. If a leak occurs in a dressing or elsewhere in the system, the therapy unit may reach its operational limit prior to its expected operational limit. For example, if the therapy unit is expected to provide negative pressure for twenty-four hours, and the mechanical assembly is consumable, a leak may prevent the therapy unit from providing negative-pressure therapy for the expected twenty-four hours. Thus, a portable therapy unit that is capable of being charged more than once may improve the provision of negative-pressure therapy for some users.

Some mechanical assemblies used to charge a portable therapy device may not be consumable, allowing the portable therapy unit to be charged multiple times. A non-consumable mechanical assembly permits the therapy unit to accommodate a leak within the therapy system by being charged a second or third time during negative-pressure therapy. However, many non-consumable mechanical assemblies require use of a component that is separable from the therapy unit. A separable component may become lost, preventing the therapy unit from reaching its operational limit. Some therapy units provide non-separable components associated with a non-consumable assembly, permitting the therapy unit to accommodate leaks within a system. Furthermore, these portable therapy units may require manual actuation that can be difficult for some patients having decreased, dexterity, strength, or skeletal issues. For example, a manually actuated therapy unit may require a user to push linearly or to pull linearly to operate the therapy unit. As the therapy unit develops increasing negative pressure, the linear compression or extension of the therapy device may require increased force to complete the action. A user having decreased muscle mass or skeletal issues may have a harder time operating the therapy device that requires the user to compress or extend a therapy unit to provide the appropriate therapy pressure. Thus, a rechargeable mechanical assembly having non-separable components may improve the provision of negative-pressure therapy for some users.

Some consumable negative-pressure therapy units can store fluid in the therapy unit. Storing fluid within the therapy unit can minimize the number of components used to provide negative-pressure therapy. Other non-consumable therapy devices cannot store fluid, necessitating an additional component or components to properly administer negative-pressure wound therapy. In both cases, the collected fluid may be trapped in a storage media such as a chamber having a desiccant or an absorbent and kept separated from the ambient environment. If the storage media is full, the consumable negative-pressure therapy device may be discarded. The addition of additional components to properly collect and store fluids from the tissue site can complicate the provision of therapy for some active or mobile users. Thus, a non-consumable therapy unit that can store fluid in the therapy unit may improve the provision of negative-pressure therapy for some users.

Often, the addition of sensors, indicators, or other devices to notify a user of therapy status can increase the cost and complexity of the therapy unit. Other therapy units use systems that required a user to be trained to appropriately read the various indicators of the therapy unit. To keep the device simple and easy to operate, some consumable and non-consumable portable therapy units do not provide an indication of the state of therapy. Many users find it difficult to comply with therapy without reminders or an easily understandable status system for the therapy unit. Thus, a therapy unit that provides easy to understand indicators of therapy status may improve negative-pressure therapy for some users.

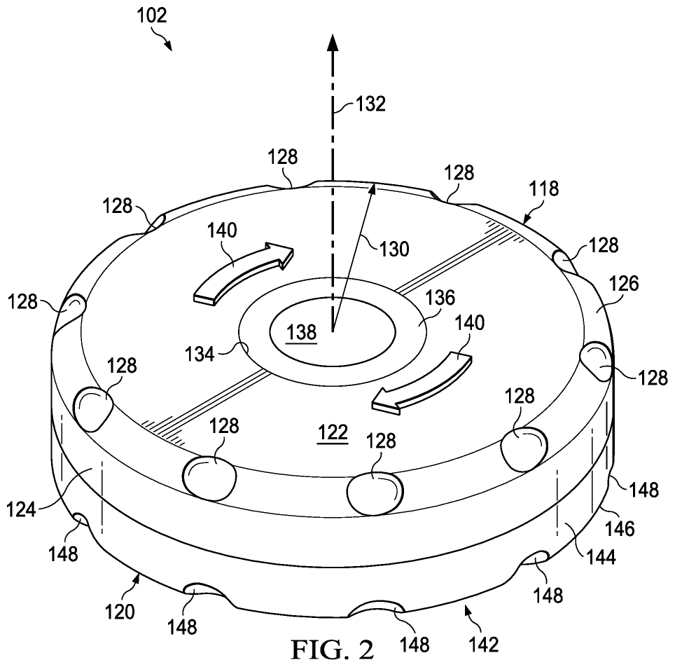

FIG. 2 is a perspective view illustrating additional details of an example embodiment of the negative-pressure source 102 of FIG. 1. The negative-pressure source 102 may be a manually-actuated negative-pressure source that may be operable by users having decreased dexterity and strength. The negative-pressure source 102 may have an increased static capacity while maintaining a size that is unobtrusive and easily charged and re-charged. Furthermore, the negative-pressure source 102 can provide assistance or reminders to patients and care givers. For example, the negative-pressure source 102 can indicate that dressing changes or therapy termination are imminent. Preferably, the reminders provide an opportunity to address the dressing change or therapy termination event without pro-longed interruption to therapy. The negative-pressure source 102 may include a bellows or foam structure that can provide a linear force and, consequently, linear generation of negative pressure. Linear generation of negative-pressure can refer to the consistent generation of a negative pressure at a specified pressure over the operation of the bellows or foam structure. In some embodiments, the negative-pressure source 102 can optionally permit fluid storage in the negative-pressure source 102.

The negative-pressure source 102 may include a casing. The casing can include one or more housing elements, such as a first housing 118 and a second housing 120. The first housing 118 may be an upper housing relative to the second housing 120, and the second housing 120 may be a lower housing relative to the first housing 118. As used herein, upper and lower describe the relative position of components in view of the figures. Use of upper and lower should not be construed as limiting the scope of the description or subsequent claims. The first housing 118 may have an end wall 122 and a side wall 124. The end wall 122 may be disc-shaped, having a radius 130 and an axis 132. The end wall 122 may have a first surface or inner surface facing a first axial direction and a second surface or outer surface facing the opposite axial direction. As used herein, inner and outer describe the relative position of components in view of the figures. Use of inner and outer should not be construed as limiting the scope of the description or subsequent claims. In some embodiments, the inner surface of the end wall 122 may face the second housing 120. The end wall 122 may be symmetrical about the axis 132. In other embodiments, the end wall 122 may not be symmetrical about the axis 132. The radius 130 may be between about 30 mm and about 80 mm. The end wall 122 may have a thickness parallel to the axis 132. Preferably, the thickness of the end wall 122 is less than the length of the radius 130. For example, the thickness of the end wall 122 can be between about 3 mm and about 8 mm. The end wall 122 may have a peripheral portion proximate to an end of the radius 130 that is opposite the axis 132.

The side wall 124 may be an annular wall coupled to the peripheral portion of the end wall 122. In some embodiments, the side wall 124 may be coupled to the end wall 122 at the end of the radius 130. In other embodiments, the side wall 124 may be coupled to the end wall 122 at a location inboard from the end of the radius 130. The side wall 124 may have a first surface or inner surface facing the axis 132 and a second or exterior surface facing away from the axis 132. The side wall 124 can have a thickness so that the inner surface has a radius from the axis 132 that is less than a radius of the outer surface from the axis 132. A shoulder may extend from the inner surface to the outer surface. The shoulder may be annular.

The end wall 122 and the side wall 124 may form an edge 126 at the location where the outer surface of the side wall 124 intersects the outer surface of the end wall 122. In some embodiments, the edge 126 may be beveled. In some embodiments, the bevel may form an internal angle between about 20 degrees and about 80 degrees with an outer surface of the end wall 122. In other embodiments, the edge 126 may be a chamfer, forming an internal angle of about 45 degrees with the outer surface of the end wall 122. As used herein, the term “about” generally refers to inclusion of a margin of error consistent with the use of the appropriate measuring apparatus to take the measurement. In some embodiments, a plurality of divots 128 may be formed in the edge 126. The plurality of divots 128 may have a circular or ovular shape having a primary diameter between about 5 mm and about 20 mm. The plurality of divots 128 may be circumferentially positioned on the edge 126 so that an arcuate distance between each divot 128 is approximately equal. In other embodiments, the divots 128 may be positioned about the edge 126 so that each divot 128 is not equally spaced from adjacent divots 128. For example, the divots 128 may be positioned to accommodate the anatomical structure of an intended user. In the illustrated embodiments, ten divots 128 are shown. In other embodiments, the negative-pressure source 102 may include no divots 128, one divot 128, or any multiple number of divots 128.

A hub 134 may be centrally disposed in the end wall 122. In some embodiments, the hub 134 may be coaxial with the axis 132 and have a radius less than the radius 130. The radius of the hub 134 may be between about 10 mm and about 40 mm. A bearing 136 may be disposed in the hub 134, and an axle 138 may be disposed in the bearing 136. The axle 138 may be cylindrical and have a radius less than the radius of the hub 134. An end of the axle 138 may be flush with the outer surface of the end wall 122. In other embodiments, the end of the axle 138 may not be flush with the outer surface of the end wall 122. The axle 138 may extend from the outer surface of the end wall 122 into the negative-pressure source 102. In some embodiments, the axle 138 may be coupled to the second housing 120. The axle 138 may be a rod, spindle, or central shaft for a rotating component. The bearing 136 can mechanically couple the hub 134 and the axle 138 and provide one or more surfaces that permit the hub 134 and the axle 138 to move relative to one another. In some embodiments, the end wall 122 may be formed without the hub 134. The end wall 122 and the side wall 124 may be rotatable about the axle 138 on the bearing 136 of the hub 134, permitting the first housing 118 to rotate on the axle relative to the second housing 120.

In some embodiments, the negative-pressure source 102 may include one or more indicators 140. The indicators 140 may be spaced radially from the hub 134 on the outer surface of the end wall 122. In some embodiments, the indicators 140 may provide an indication of the preferential direction of rotation of the first housing 118 on the axle 138. The indicators 140 may comprise arrows having a head facing a desired direction of rotation of the first housing 118. In other embodiments, the indicators 140 may be other visual or written symbols of the desired direction of rotation.

The second housing 120 may have an end wall 142 and a side wall 144. The end wall 142 may be disc-shaped. In some embodiments, the end wall 142 may be co-axial with the end wall 122 on axis 132. The end wall 142 may have a first surface or inner surface facing a first axial direction and a second surface or outer surface facing the opposite axial direction. In some embodiments, the inner surface may face the first housing 118. The end wall 142 may be symmetrical about the axis 132. In other embodiments, the end wall 142 may not be symmetrical about the axis 132. The end wall 142 may have a thickness parallel to the axis 132. In some embodiments, the thickness of the end wall 142 can be between about 2 mm and about 3 mm.

The side wall 144 may be an annular wall coupled to a peripheral portion of the end wall 142. In other embodiments, the side wall 144 may be coupled to the end wall 142 at a location inboard from the peripheral portion of the end wall 142. The side wall 144 may have a first surface or inner surface facing the axis 132 and a second or outer surface facing away from the axis 132. The side wall 144 can have a thickness so that the inner surface has a radius from the axis 132 that is less than a radius of the outer surface from the axis 132. In some embodiments, the thickness of the side wall 144 can be between about 2 mm and about 3 mm. A shoulder may extend from the inner surface to the outer surface. The shoulder may be annular.

The end wall 142 and the side wall 144 may form an edge 146 at the location where the outer surface of the side wall 144 intersects the outer surface of the end wall 142. In some embodiments, the edge 146 may be beveled. In some embodiments, the bevel may form an internal angle between about 20 degrees and about 80 degrees with an outer surface of the end wall 142. In other embodiments, the edge 146 may be a chamfer, forming an internal angle of about 45 degrees with the outer surface of the end wall 142. In some embodiments, a plurality of divots 148 may be formed in the edge 146. The plurality of divots 148 may have a circular or ovular shape having a primary diameter between about 5 mm and about 20 mm. The plurality of divots 148 may be circumferentially positioned on the edge 146 so that an arcuate distance between each divot is approximately equal. In other embodiments, the divots 148 may be positioned about the edge 146 so that each divot 148 is not equally spaced from adjacent divots 148. For example, the divots 148 may be positioned to accommodate the anatomical structure of an intended user. In the illustrated embodiments, ten divots 148 are shown. In other embodiments, the negative-pressure source 102 may include no divots 148, one divot 148, or any multiple number of divots 148.

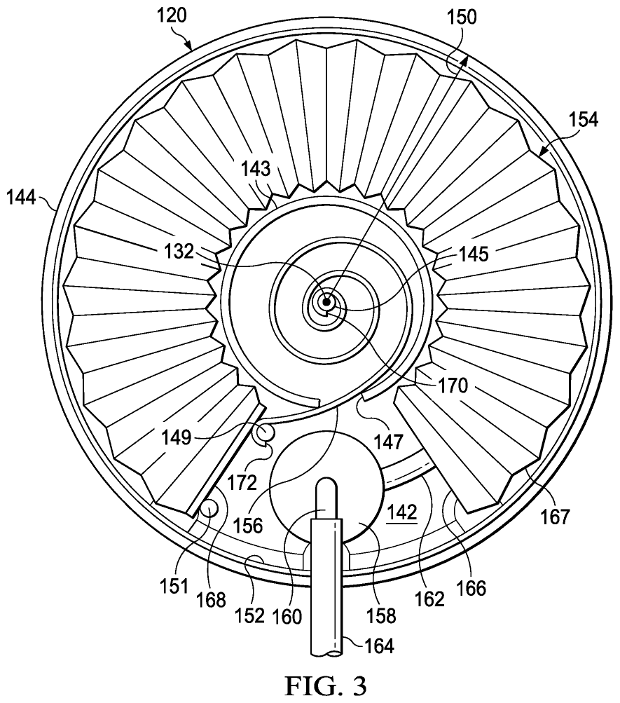

FIG. 3 is a plan view illustrating additional details that may be associated with some embodiments of the second housing 120. The end wall 142 may have a radius 150 substantially equal to the radius 130 of the end wall 122. The peripheral portion of the end wall 142 may be disposed at an end of the radius 150 opposite the axis 132. The side wall 144 may extend from the end wall 142 parallel to the axis 132 to form a cavity 152. The cavity 152 may have a depth substantially equal to a height of the side wall 144. In some embodiments, the depth may be between about 20 mm and about 40 mm. A support 145 may be disposed in the cavity 152. In some embodiments, the support 145 may be a column that is coaxial with the axis 132 and coupled to the axle 138. The support 145 may have a radius equal to a radius of the axle 138. In other embodiments, the support 145 may have a radius less than the radius of the axle 138. A wall 143 may also be disposed within the cavity 152. The wall 143 may be an annular wall circumscribing the axis 132 and having an inner surface radially spaced from the axis 132. In some embodiments, an inner surface of the wall 143 may be between about 1 mm and about 2 mm from an outer surface of the support 145. In some embodiments, the wall 143 may have an opening 147. The opening 147 may comprise a slot or other slender opening. The opening 147 can be positioned circumferentially at a clock position associated with about six o'clock.

A negative-pressure reservoir 154, a biasing member 156, and a regulator 158 may be disposed in the cavity 152. The regulator 158 may be a type of controller 112 and be positioned in a first position within the cavity 152. For example, the regulator 158 may be disposed at the six o'clock position proximate to the opening 147 in the wall 143 as illustrated in FIG. 3. The regulator 158 may be coupled to the end wall 142. In some embodiments, the regulator 158 may have a therapy port 160 and a suction port 162. The therapy port 160 may be adapted to be fluidly coupled to a tube 164 or another fluid conductor. The tube 164 may further be fluidly coupled to a dressing, such as the dressing 104, or other therapy device. The suction port 162 can be a tube or other fluid conductor, and the suction port 162 may be fluidly coupled to the negative-pressure reservoir 154. The regulator 158 can be fixed to the end wall 142 within the cavity 152, preventing the regulator 158 from moving with rotation of the first housing 118 relative to the second housing 120.

The negative-pressure reservoir 154 may have a first end 166 and a second end 168. The negative-pressure reservoir 154 may have a reservoir wall 167 extending between the first end 166 and the second end 168. The reservoir wall 167 may be an annular wall forming a cavity bounded by the first end 166, the reservoir wall 167, and the second end 168. The first end 166 of the negative-pressure reservoir 154 may be affixed to the end wall 142 and spaced an arcuate distance from the regulator 158. For example, the first end 166 of the negative-pressure reservoir 154 may be positioned at the five o'clock position as illustrated in FIG. 3. The suction port 162 can be fluidly coupled to the first end 166 of the negative-pressure reservoir 154. For example, a tube or other fluid coupler may fluidly couple the suction port 162 to the first end 166. In some embodiments, at least one of the suction port 162 or the first end 166 can have a one-way valve disposed therein. For example, a check valve may be positioned in the suction port 162 to permit fluid to flow from the regulator 158 into the first end 166 of the negative-pressure reservoir 154.

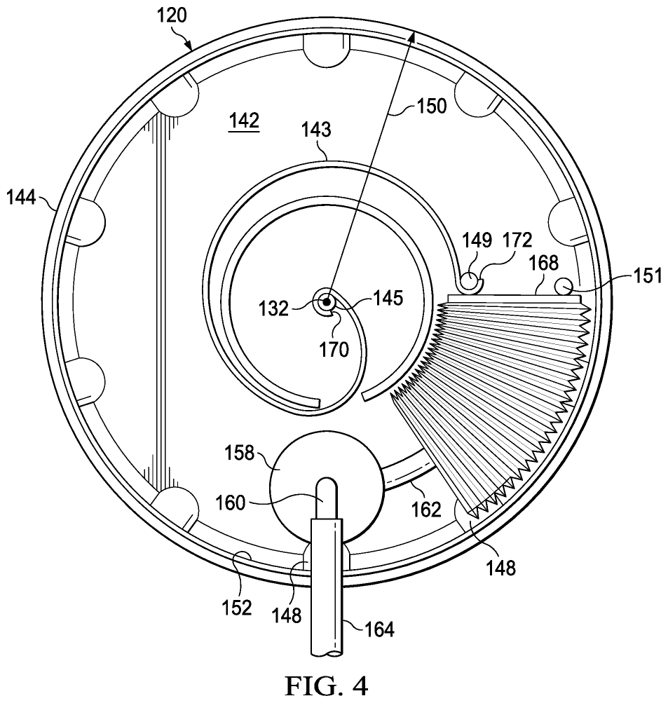

The second end 168 can be disposed on an opposite end of the reservoir wall 167 from the first end 166. The second end 168 of the negative-pressure reservoir 154 may be free relative to the first end 166. The second end 168 may have a one-way valve disposed therein. For example, the second end 168 may have a check valve positioned in the second end 168 to permit fluid to flow from the negative-pressure reservoir 154 into the ambient environment. In some embodiments, the second end 168 may have a coupling pin 149. The coupling pin 149 may be fixed to the second end 168 so that the coupling pin 149 and the second end 168 may move as a single body. The coupling pin 149 may be a cylindrical body coupled to the second end 168 to create a gap between a side surface of the coupling pin 149 and a surface of the second end 168. In other embodiments, the coupling pin 149 may be another attachment device, such as an adhesive, a weld, a screw, a nail, or other pin. The coupling pin 149 may be further coupled to the inner surface of the end wall 122 of the first housing 118. In some embodiments, the second end 168 of the negative-pressure reservoir 154 may be moveable relative to the first end 166. For example, the second end 168 may be moveable an arcuate distance from the first end 166 between a first position, illustrated in FIG. 3, and a second position. In some embodiments, the second end 168 may move through 2π radians from the first end 166. In the first position, the second end 168 may be positioned at an approximately seven o'clock position relative to the regulator 158. In the second position, illustrated in FIG. 4, the second end 168 can be positioned at an approximately three o'clock position relative to the regulator 158. The second end 168 can move clockwise from the first position to the second position and counterclockwise back to the first position.

In some embodiments, a projection 151 may be operatively coupled to the second end 168 of the negative-pressure reservoir 154. The projection 151 can be a solid body coupled to the inner surface of the end wall 122 and extending toward the second housing 120. The projection 151 may have a strength sufficient to resist a moment force exerted on the projection 151 in response to rotation of the first housing 118 relative to the second housing 120. The projection 151 may rotate as the first housing 118 rotates. As the projection 151 rotates, the projection 151 may exert a force on the second end 168 of the negative-pressure reservoir 154 that urges the second end 168 toward the first end 166.

In some embodiments, the reservoir wall 167 is flexible. For example, the reservoir wall 167 may be corrugated, being shaped into alternating ridges and grooves that permit the reservoir wall 167 to be contracted and expanded between the first position, illustrated in FIG. 3, and the second position, illustrated in FIG. 4. The first position may also be referred to as an expanded position, and the second position may also be referred to as a collapsed position. In some embodiments, the negative-pressure reservoir 154 may be a bellows. A bellows can be a flexible bag having rigid or semi-rigid ends. The flexible bag may form an airtight cavity that can be expanded and contracted by moving the ends relative to one another. A bellows may have one or more valves to permit fluid to move in one direction through the bellows. For example, a bellows may have a first valve configured to permit fluid to flow into the bellows through an inlet and prevent fluid from flowing out of the bellows through the inlet. A bellows may have a second valve configured to permit fluid to flow out of the bellows through the outlet and prevent fluid from flowing into the bellows through the outlet.

Movement of the second end 168 relative to the first end 166 can expand and contract the volume formed by the cavity of the negative-pressure reservoir 154, drawing fluid into and forcing fluid out of the negative-pressure reservoir 154. The negative-pressure reservoir 154 may be coupled to the regulator 158 so that the negative-pressure reservoir 154 may draw fluid from the regulator 158 into the negative-pressure reservoir 154 as the negative-pressure reservoir 154 expands to the first position illustrated in FIG. 3

The biasing member 156 may be disposed within the cavity 152 and have a first end 170 and a second end 172. The biasing member 156 may be disposed between the wall 143 and the support 145. In some embodiments, the biasing member 156 may circumscribe the support 145. The first end 170 may be fixed. For example, the first end 170 may be coupled to the support 145. The second end 172 of the biasing member 156 may be a free end. For example, the second end 172 may be coupled to the second end 168 of the negative-pressure reservoir 154. In some embodiments, the second end 172 may be coupled to the second end 168 through the coupling pin 149. The second end 172 of the biasing member 156 may pass through the opening 147. For example, the opening 147 may be positioned proximate to the regulator 158, and the second end 172 of the biasing member 156 may pass through the opening 147 to couple to the second end 168 of the negative-pressure reservoir 154. In some embodiments, the biasing member 156 may be a ribbon spring or similar device capable of providing a constant force. For example, the biasing member 156 can be a constant-force spring. The constant force spring may be a spring for which the force the spring exerts over its range of motion is generally constant. The constant-force spring may be constructed from a rolled ribbon of spring steel or similar having a first end fixed, for example, the first end 170 fixed to the support 145, and the second end 172 being free. The second end 172 can be extended to generate a spring force. In some embodiments, other springs may be used. A spring constant of the biasing member 156 may be selected for a desired therapeutic pressure of the negative-pressure reservoir 154. Generally, the biasing member 156 can be selected based on the desired therapeutic pressure and any friction forces that may resist the expansion of the negative-pressure reservoir 154. The biasing member 156 may further bias the second end 168 of the negative-pressure reservoir 154 to the first position illustrated in FIG. 3. Generally, the biasing member 156 may be at rest in the first position illustrated in FIG. 3.

In some embodiments, the corrugations of the reservoir wall 167 may cause the reservoir wall 167 to function similar to the biasing member 156. As a result, the reservoir wall 167 may have a spring force that biases the reservoir wall 167 to the first position illustrated in FIG. 3. Where the spring force of the reservoir wall 167 is sufficiently high, the biasing member 156 can be eliminated. Preferably, the spring force of the reservoir wall 167 can be selected to generate a linear force and a linear negative-pressure over the full expansion of the negative-pressure reservoir 154.

FIG. 4 is a plan view illustrating additional details that may be associated with some embodiments of the second housing 120 of FIG. 1. As illustrated in FIG. 4, the biasing member 156 and the negative-pressure reservoir 154 are in the second position. The second end 172 of the negative-pressure reservoir 154 is moved through an arc, bringing the second end 172 proximate to the first end 170. In the second position, the volume of the cavity formed by the negative-pressure reservoir 154 is decreased, forcing fluid in the cavity out of the cavity. For example, fluid may flow out of the second end 168 of the negative-pressure reservoir 154 through the check valve associated with the second end 168.

In the second position, the second end 172 of the biasing member 156 is displaced from the rest position of FIG. 3. Movement into the second position stretches the biasing member 156, generating a spring force that urges the biasing member 156 to draw the second end 172 back to the first position illustrated in FIG. 3. As the biasing member 156 draws the second end 172 and the second end 168 back to the first position, the cavity of the negative-pressure reservoir 154 increases in volume, drawing air into the negative-pressure reservoir 154 through the suction port 162 from the regulator 158, which in turn can draw fluid from another device coupled to the therapy port 160, for example, the dressing 104.

In operation, the first housing 118 may be rotated relative to the second housing 120. The rotational motion of the first housing 118 may be translated to the second end 168 of the negative-pressure reservoir 154 through the coupling pin 149 and/or the projection 151. As the first housing 118 rotates relative to the second housing 120, the second end 168 may be rotated toward the first end 166 of the negative-pressure reservoir 154, forcing fluid out of the negative-pressure reservoir 154. As the second end 168 of the negative-pressure reservoir 154 rotates toward the first end 166, the second end 168 will draw the second end 172 of the biasing member 156 toward the first end 166 of the negative-pressure reservoir 154, extending the biasing member 156 and generating a spring force urging the second end 172 of the biasing member 156 back to the first position illustrated in FIG. 3. As the biasing member 156 draws the second end 172 and the second end 168 back to the first position, the volume of the negative-pressure reservoir 154 will increase, generating a negative pressure in the negative-pressure reservoir 154 that can be fluidly communicated to another device, such as the regulator 158 or the dressing 104. The biasing member 156, being a constant-force spring, may generate a linear pressure profile in the negative-pressure reservoir 154. A linear pressure profile may permit steadier application of negative-pressure therapy having fewer variations in the therapy pressure at the tissue site.

In some embodiments, movement of the negative-pressure reservoir 154 may be constrained by the wall 143 and the side wall 144. The negative-pressure reservoir 154 may expand and contract, being constrained between the wall 143 and the side wall 144 to prevent undesired motion of the reservoir wall 167. While described with respect to rotation of the first housing 118 and the negative-pressure reservoir 154 clockwise to charge the negative-pressure source 102 and counterclockwise to discharge the negative-pressure source 102, the preferred direction of rotation can be changed without changing the operating principles described herein.

In some embodiments, the negative-pressure reservoir 154 may have a static capacity of about 60 milliliters. The negative-pressure reservoir 154 may be capable of receiving and storing fluid from the tissue site. For example, the negative-pressure reservoir 154 may have an absorbent or desiccant stored therein. Fluid from the tissue site may flow into the negative-pressure reservoir 154 where liquids can be trapped by the absorbent. In some embodiments, an absorbent can be disposed in an internal surface of the negative-pressure reservoir 154. For example, the absorbent can be coated to the reservoir wall 167 of an interior of the negative-pressure reservoir 154. In some embodiments, the absorbent can be positioned to prevent movement of the absorbent, prevent blockage of the negative-pressure reservoir 154, and avoid interference with expansion of the negative-pressure reservoir 154.

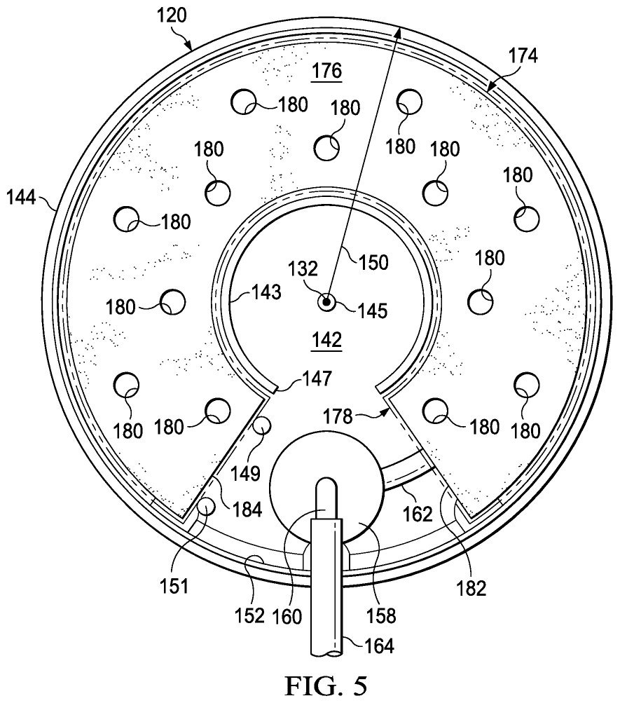

FIG. 5 is a schematic top view illustrating additional details of another embodiment of the second housing 120. In some embodiments, the negative-pressure reservoir 154 and the biasing member 156 can be combined. For example, the negative-pressure reservoir 154 and the biasing member 156 can be a foam reservoir 174. The foam reservoir 174 may include a foam block 176 and an envelope 178. In some embodiments, the foam block 176 may be an open-cell reticulated foam. The open-cell reticulated foam can be felted. In some embodiments, the open-cell reticulated foam can be felted to have a compression of about 3:1 to 5:1. For example, the open-cell reticulated foam can be compressed to have a density between about 3 times and about 5 times its original density. In some embodiments, the foam block 176 can include a plurality of perforations 180. The plurality of perforations 180 can extend completely through the foam block 176. In other embodiments, the plurality of perforations 180 may not extend through the foam block 176. Each of the plurality of perforations may have a pitch and a location selected to generate a linear force. For example, each perforation 180 can have a diameter between about 3 mm and about 6 mm and a pitch between adjacent perforations 180 between about 5 mm and about 10 mm. In some embodiments, the foam block 176 may have a void space volume of about 60 milliliters (mL). A void space volume can refer to the total volume of the foam block 176 that is free of solid material. For example, an open-cell reticulated foam can have a plurality of pores that are free of solid material. The plurality of pores form a portion of the volume of the foam, which can be a substantial portion, but typically less than the total volume of the foam. The void space volume of the foam block 176 can be increased by the plurality of perforations 180 so that the total void space volume of the foam block 176 is greater than the void space volume of the pores of the foam block 176 alone. In some embodiments, about 50% and about 80% of the volume of the foam block 176 may be the void space volume.

The envelope 178 can surround the foam block 176 so that the foam block 176 is encapsulated in the envelope 178. Preferably, the foam block 176 is sealed from the ambient environment within the envelop 178. The envelope 178 be formed from a polyurethane or polyethylene material. In some embodiments, the envelope 178 may be flexible and conform to the foam block 176. In some embodiments, the envelope 178 may be formed from a polyurethane film having a thickness of about 100 microns. In other embodiments, the envelope 178 can be a coating applied to the foam block 176. For example, the foam block 176 can be coated with polyurethane, polyethylene, silicone, or polyvinyl chloride, sealing the void space volume of the foam block 176 from the ambient environment.

The foam reservoir 174 can have a first end 182 and a second end 184. The first end 182 of the foam reservoir 174 may be affixed to the end wall 142 and spaced an arcuate distance from the regulator 158. For example, the first end 182 of the foam reservoir 174 may be positioned at the five o'clock position as illustrated in FIG. 5. The suction port 162 can be fluidly coupled to the first end 182 of the foam reservoir 174. For example, a tube or other fluid coupler may fluidly couple the suction port 162 to the first end 182. In some embodiments, at least one of the suction port 162 or the first end 182 can have a one-way valve disposed therein. For example, a check valve may be positioned in the suction port 162 to permit fluid to flow from the regulator 158 into the first end 182 of the foam reservoir 174.

The second end 184 can be disposed on an opposite end of the foam reservoir 174 from the first end 182. The second end 184 of the foam reservoir 174 may be free relative to the first end 182. The second end 184 may have a one-way valve disposed therein. For example, the second end 184 may have a check valve positioned in the second end 184 to permit fluid to flow from the foam reservoir 174 into the ambient environment. In some embodiments, the coupling pin 149 can be coupled to the second end 184. The coupling pin 149 may be fixed to the second end 184 so that the coupling pin 149 and the second end 184 may move as a single body. In some embodiments, the projection 151 can be operatively coupled to the second end 184 of the foam reservoir 174. In some embodiments, the second end 184 of the foam reservoir 174 may be moveable relative to the first end 182. For example, the second end 184 may be moveable an arcuate distance from the first end 182 between a first position, illustrated in FIG. 5, and a second position. In some embodiments, the second end 184 may move through 2π radians from the first end 182. In other embodiments, the second end 184 may move through less than 2π radians from the first end 182. In the first position, the second end 184 may be positioned at an approximately seven o'clock position relative to the regulator 158.

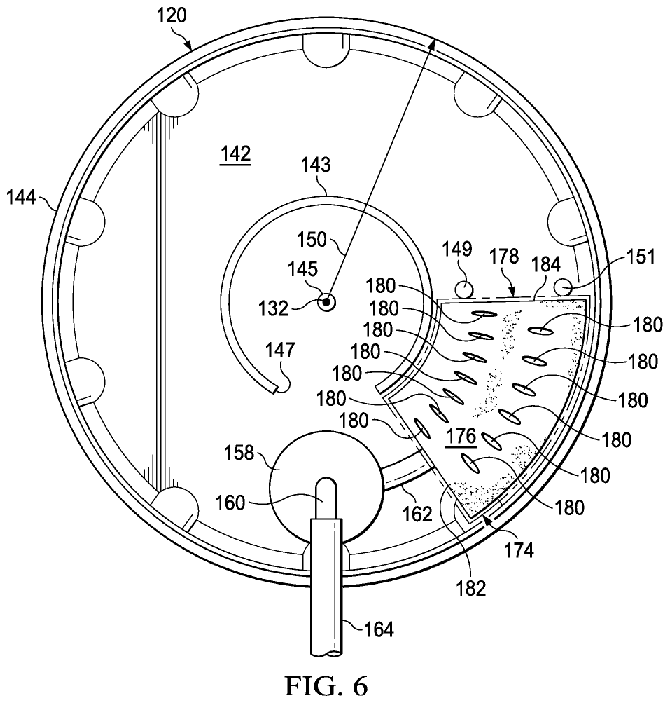

FIG. 6 is a schematic top view illustrating additional details of the second housing 120 of FIG. 5. In the second position, illustrated in FIG. 6, the second end 184 can be positioned at an approximately three o'clock position relative to the regulator 158. The second end 184 can move clockwise from the first position to the second position and back to the first position. The first position may also be referred to an expanded position, and the second position may also be referred to as a collapsed position.

Movement of the second end 184 relative to the first end 182 can expand and contract the volume formed by the cavity of the foam reservoir 174, drawing fluid into and forcing fluid out of the foam reservoir 174. The foam reservoir 174 may be coupled to the regulator 158 so that the foam reservoir 174 may draw fluid from the regulator 158 into the foam reservoir 174 as the foam reservoir 174 expands to the first position illustrated in FIG. 5.

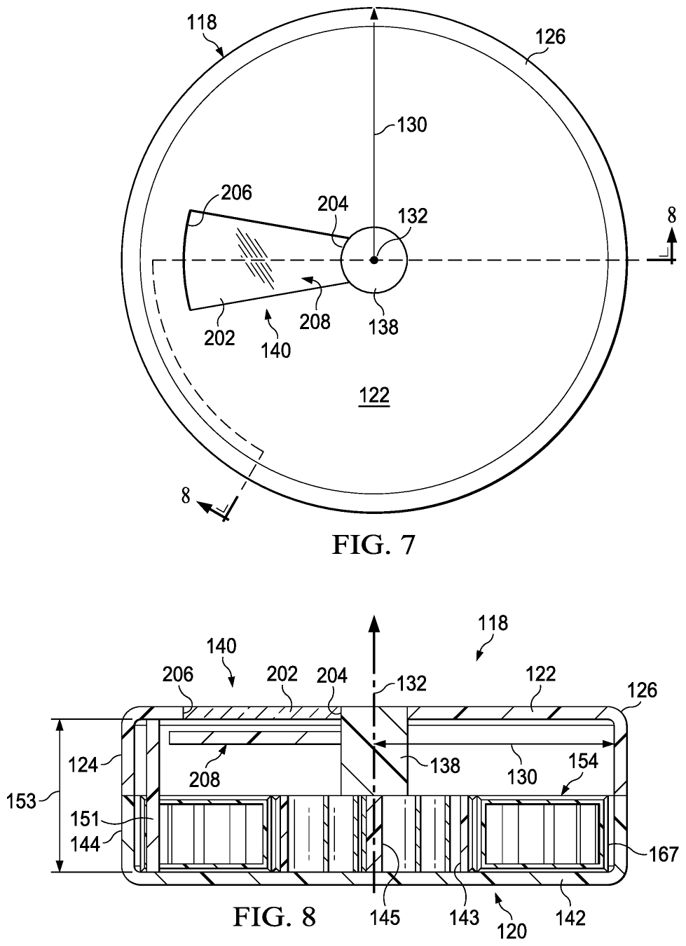

FIG. 7 is a schematic top view illustrating additional details of another embodiment of the first housing 118. The first housing 118 may include one or more indicators 140. In some embodiments, the indicator 140 may include a window 202 and an indicator disc 208. The indicator disc 208 can be a type of charge status indicator or life status indicator. The window 202 may extend through the end wall 122 from the outer surface to the inner surface of the end wall 122. A first end 204 of the window 202 may be disposed proximate to the axle 138, and a second end 206 of the window 202 may be disposed proximate to the edge 126. Sides of the window 202 may extend from the first end 204 to the second end 206 parallel to the radius 130. In some embodiments, the window 202 may include a transparent material, such as glass or plastic, disposed in the window 202, permitting visual inspection through the end wall 122 and preventing physical passage across the end wall 122.

FIG. 8 is a sectional view illustrating additional details of the first housing 118 and the indicator 140. The indicator disc 208 may be disposed between the end wall 122 and the end wall 142. The indicator disc 208 may be disc-shaped. In some embodiments, the indicator disc 208 can be coupled to the axle 138 proximate to the end wall 122. The indicator disc 208 may be fixed relative to the end wall 122 and be visible through the window 202. For example, the end wall 122 can rotate on the axle 138. The indicator disc 208 being coupled to the axle 138 may be stationary relative to the end wall 122.

The first housing 118 can include the projection 151. The projection 151 may be cylindrical or columnar and have a length 153. The length 153 can be greater than a height of the side wall 124 so that the projection 151 protrudes from the first housing 118 toward the second housing 120 The projection 151 can engage the second end 168 of the negative-pressure reservoir 154. The projection 151 may be positioned in the seven o'clock position if the negative-pressure reservoir 154 is in the first position.

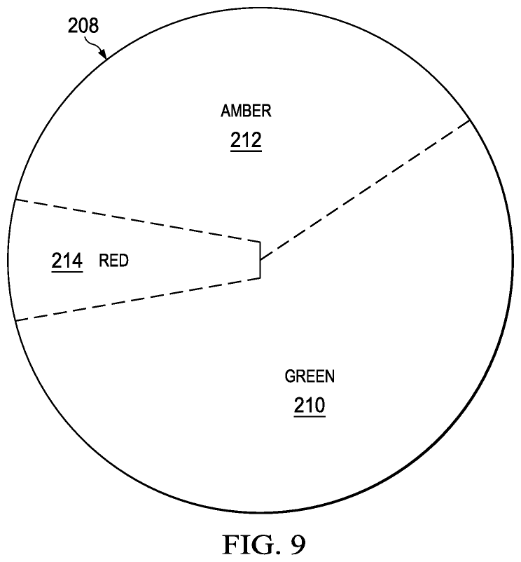

FIG. 9 is a top view illustrating additional details of the indicator disc 208. The indicator disc 208 may have a first indicator 210, a second indicator 212, and a third indicator 214. In some embodiments, the first indicator 210, the second indicator 212, and the third indicator 214 can be portions of a surface of the indicator disc 208. For example, the first indicator 201, and the second indicator 212, and the third indicator 214 can be potions of the surface of the indicator disc 208 having different colors. Preferably, the first indicator 210, the second indicator 212, and the third indicator 214 are disposed on the surface of the indicator disc 208 facing the end wall 122. The first indicator 210, the second indicator 212, and the third indicator 214 may not be equal portions of the indicator disc 208. For example, the first indicator 210 may be about two-thirds of the surface of the indicator disc 208; of the remaining one-third, the second indicator 212 may be about two-thirds of the surface of the indicator disc 208, and the third indicator 214 may cover the remainder of the surface of the indicator disc 208. In other embodiments, the proportions of the first indicator 210, the second indicator 212, and the third indicator 214 on the surface of the indicator disc 208 may be different.

The first indicator 210, the second indicator 212, and the third indicator 214 may indicate a charge status or expected life of the negative-pressure source 102. For example, the first indicator 210 may indicate that the negative-pressure source 102 is charged or that the expected life of the negative-pressure source 102 is normal. The second indicator 212 may indicate that the negative-pressure source 102 is nearing the end of its charge or that the expected life of the negative-pressure source 102 is nearing its end. The third indicator 214 may indicate that the negative-pressure source 102 is no longer charged or that the life of the negative-pressure source 102 is over. As shown in FIG. 9, the first indicator 210 is green, the second indicator 212 is amber, and the third indicator 214 is red. Other colors may be used for the first indicator 210, the second indicator 212, and the third indicator 214.

In operation, the negative-pressure source 102 may be in an uncharged state. An uncharged state can be an operating state of the negative-pressure source 102 where the negative-pressure source 102 is not providing negative-pressure therapy. In the uncharged state, the window 202 can be positioned over the third indicator 214. The negative-pressure source can be moved from an uncharged state to a charged state where the negative-pressure source 102 can provide negative-pressure therapy. To move the negative-pressure source 102 from the uncharged state to the charged state, the first housing 118 can be rotated relative to the second housing 120. As the first housing 118 is rotated on the axle 138, the window 202 may be rotated from the third indicator 214 over the second indicator 212 and the first indicator 210. Similarly, as the first housing 118 rotates on the axle 138, the projection 151 can move the second end 168 of the negative-pressure reservoir 154 from the first position illustrated in FIG. 3 to the second position illustrated in FIG. 4, compressing the negative-pressure reservoir 154. If the negative-pressure reservoir 154 is in the second position, the window 202 can be disposed over the first indicator 210. As the biasing member 156 draws the second end 168 of the negative-pressure reservoir 154 toward the first position illustrated in FIG. 3, the window 202 can be moved in conjunction through the coupling of the first housing 118 to the projection 151. In some embodiments, the first indicator 210, the second indicator 212, and the third indicator 214 can be associated with the remaining static fluid capacity of the negative-pressure reservoir 154 to indicate to a user that the therapy being provided is at or close to termination. In some embodiments, the first indicator 210, the second indicator 212, and the third indicator 214 can indicate the remaining capacity of the negative-pressure reservoir 154. For example, if liquid is drawn into the negative-pressure reservoir 154, the first indicator 210, the second indicator 212, and the third indicator 214 can indicate the remaining free space within the negative-pressure reservoir 154.

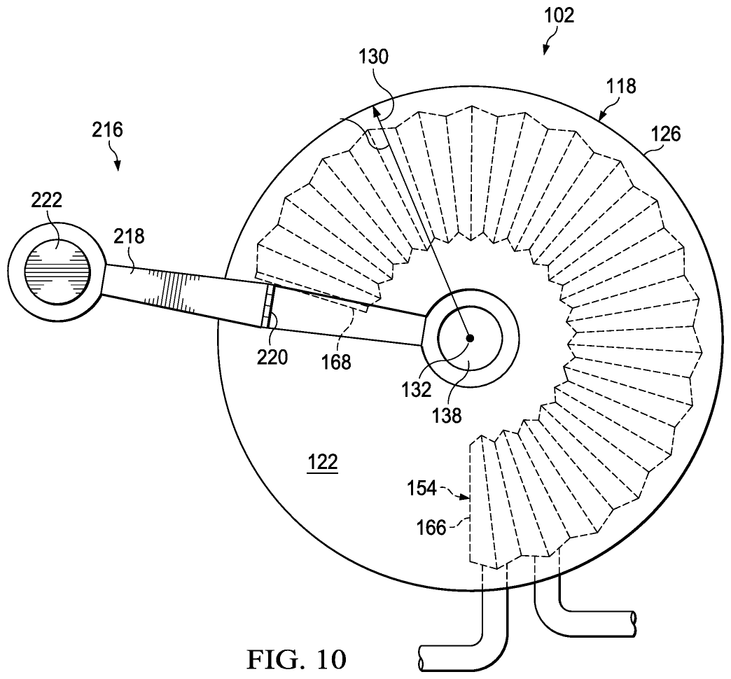

FIG. 10 is a schematic plan view illustrating additional details of the negative-pressure source 102. In some embodiments, the first housing 118 may include a rotation assembly 216. The rotation assembly 216 can include a rotation arm 218, a hinge 220, and an attachment device, such as a snap 222. The hinge 220 may be a mechanical bearing connecting the end wall 122 and the rotation arm 218. The hinge 220 may have an axis of rotation that is parallel to a tangent of the end wall 122 and allows one degree of freedom. In some embodiments, the hinge 220 can be disposed proximate to the edge 126, and the hinge 220 may be capable of rotation within a plane defined by the radius 130 and the axis 132. The hinge 220 may be coupled to the outer surface of the end wall 122 of the first housing 118. In some embodiments, the rotation arm 218 may be coupled to the hinge 220. The rotation arm 218 can be a solid body, such as a beam or arm, having a first end and a second end. Generally, the rotation arm 218 may be longer than it is wide. The first end of the rotation arm 218 can be coupled to the hinge 220. If the hinge 220 is open, the rotation arm 218 may extend from the hinge 220 past the edge 126. In some embodiments, the snap 222 can be coupled to the second end of the rotation arm 218. The snap 222 can be a type of mechanical fastener, such as a pin, latch, interference fit component, or other mechanically securable device. The snap 222 may have a first component secured to the second end of the rotation arm 218 and a second component secured to the axle 138. In some embodiments, the rotation arm 218 can be rotated on the hinge 220 so that the snap 222 is disposed over the axle 138. The snap 222 can then be releaseably coupled to the axle 138. If extended, as shown in FIG. 10, the rotation arm 218 can be used to provide additional leverage for the rotation of the first housing 118 relative to the second housing 120.

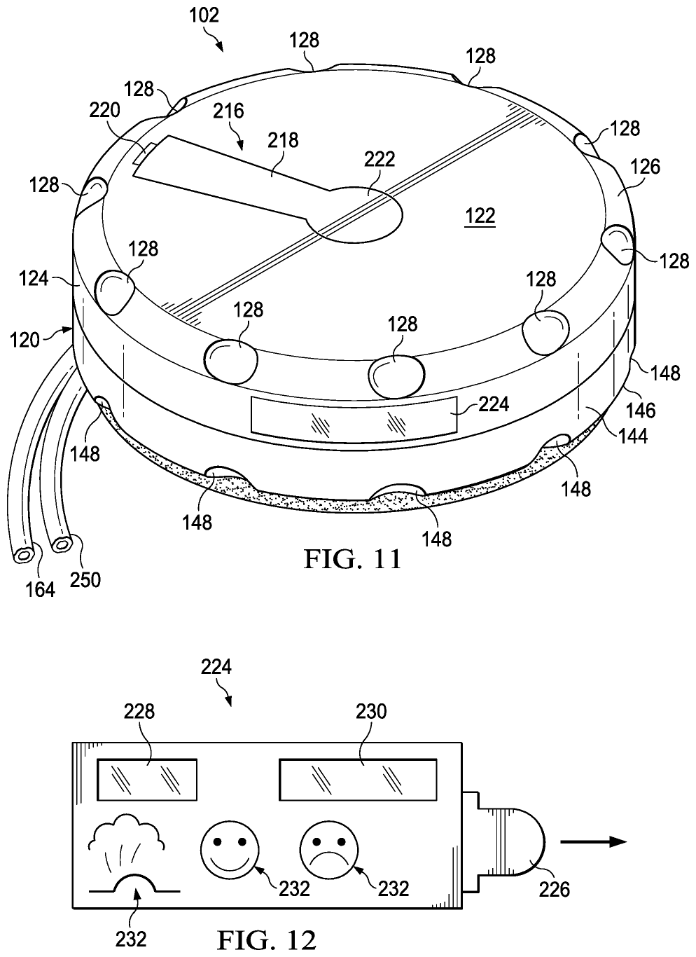

FIG. 11 is a perspective view illustrating additional details of the negative-pressure source 102. As illustrated in FIG. 11, the hinge 220 can be closed, rotating the rotation arm 218 in and positioning the snap 222 over the axle 138. The snap 222 can be secured to the axle 138. In some embodiments, the end wall 122 may have a recess to receive the hinge 220, the rotation arm 218, and the snap 222. If the hinge 220 is closed, the rotation arm 218 and the snap 222 may be flush with the outer surface of the end wall 122.

In some embodiments, the indicator 140 may comprise a digital indicator 224. The digital indicator 224 may be an embodiment of the controller 112 and can be another type of charge status indicator or life status indicator. The digital indicator 224 can include or be coupled to one or more sensors, such as the first sensor 114 and the second sensor 116, to determine a status of the negative-pressure source 102, for example, a duration of therapy, a pressure provided, or other information related to the negative-pressure source 102 and the provision of negative-pressure therapy. The digital indicator 224 may be coupled to the side wall 124 of the first housing 118. In other embodiments, the digital indicator 224 may be coupled to other components of the negative-pressure source 102. The digital indicator 224 may be an electrically powered module having integrated indicators and pressure monitoring. In some embodiments, the digital indicator 224 may be activated by the rotation of the first housing 118 relative to the second housing 120. In some embodiments, the negative-pressure source 102 may include a conduit 250. The conduit 250 can be a tube or other fluid conductor. The conduit 250 may be fluidly coupled to the digital indicator 224 and further fluidly coupled to the dressing 104. The conduit 250 can fluidly communicate between the dressing 104 and the digital indicator 224. In other embodiments, the negative-pressure source 102 may include a multi-lumen conduit combing the tube 164 and the conduit 250.

FIG. 12 is a schematic view illustrating additional details of the digital indicator 224 of FIG. 11. The digital indicator 224 may include a first display 228, a second display 230, and one or more therapy indicators 232. In some embodiments, the digital indicator 224 may be an electrically powered device. For example, the digital indicator 224 may include a battery that powers one or more electrical components of the digital indicator 224. In some embodiments, a pull-to-start insulator 226 can be used to prevent drains from the battery prior to use of the negative-pressure source 102. The pull to start insulator 226 can be an insulated tab disposed between the battery and an electrical contact that completes an electrical circuit powering the digital indicator 224. The insulator 226 may interrupt the power circuit at the location of the battery. The insulator 226 may have a first end disposed between a positive or negative terminal of the battery and the circuit to which the battery is electrically coupled. The insulator 226 may have a second end coupled to the second housing 120, for example, the side wall 144. If the first housing 118 is rotated relative to the second housing 120, the rotation may pull the insulator 226 from the circuit, powering the digital indicator 224.

The digital indicator 224 may provide information or data to the user about the status of the pump and the tissue site. In some embodiments, the digital indicator 224 may include a sensor, such as the first sensor 114 or the second sensor 116. The sensor can be a pressure sensor that is fluidly connected to the wound site via the conduit 250. The digital indicator 224 may include a data storage device, such as memory, that can store data read by the sensor. The digital indicator 224 may permit the data to be extracted for analysis by a caregiver. The first display 228, the second display 230, and the one or more therapy indicators 232 can be used to motivate a user to charge and re-charge the negative-pressure source 102 and to log how often the negative-pressure source 102 is re-charged and a flow/leak rate. The first display 228 and the second display 230 can be liquid crystal displays (LCDs). In some embodiments, the digital indicator 224 may provide a graph of the last twelve hours wound pressure to either the first display 228, the second display 230, or both. In some embodiments, the digital indicator 224 may include a USB, wireless internet, near-field communication (NFS), or Bluetooth connection that can communicated data collected by the digital indicator to another device, such as a computer or smart phone.

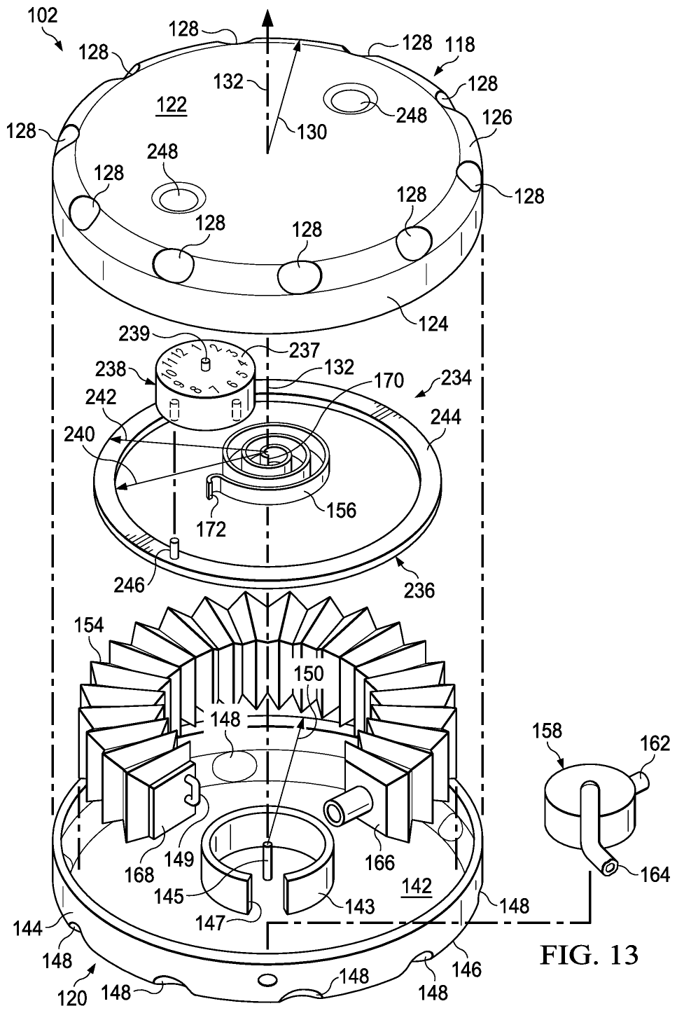

FIG. 13 is a perspective view illustrating additional details of another embodiment of the negative-pressure source 102. In some embodiments, the indicators 140 may comprise an indicator system 234. The indicator system 234 may include a ring 236 and a first counter 238. The ring 236 may be a ring having first radius 240 from the axis 132 and a second radius 242 from the axis 132. The second radius 242 may be larger than the first radius 240, forming a shoulder 244. In some embodiments, the second radius 242 may be between about 5 mm and about 10 mm larger than the first radius 240. The shoulder 244 may be between the first radius 240 and the second radius 242. The shoulder 244 may be annular. In some embodiments, the ring 236 may be coupled to the side wall 144 between the negative-pressure reservoir 154 and the end wall 122. For example, an edge of the ring 236 formed by the depth of the ring 236 at the second radius 242 may be coupled to the inner surface of the side wall 144. In some embodiments, the ring 236 may be fixed to the side wall 144, preventing relative motion between the side wall 144 and the ring 236. For example, the ring 236 may be welded, bonded, adhered, or otherwise secured to the side wall 144. The ring 236 may include a tab 246. The tab 246 may be a cylindrical body having a first end coupled to the shoulder 244 and a second end spaced apart from the shoulder 244. In some embodiments, the tab 246 may be fixed to the ring 236.

The first counter 238 can be another type of charge status indicator or life status indicator and may be a clockwork or a timer operative to determine a timed interval and display the current time relative to the interval. A clockwork can be a mechanical mechanism having a motor, for example a clockwork motor. The motor can include a mainspring capable of storing energy. Often, energy can be stored in the mainspring by winding the motor. The motor can then drive a plurality of gears. A motor can also include an electrically powered device capable of driving a plurality of gears. In the case of a timer, the clockwork may include a balance wheel and a train gear or a fan fly to parse out regular intervals of time. The clockwork may be capable of marking increasing or decreasing intervals of time. In some embodiments, the first counter 238 may be operable to track a time period of twelve days or twelve twenty-four hour periods. The first counter 238 can be coupled to the first housing 118. In some embodiments, the first counter 238 can be coupled to the inner surface of the end wall 122. The first counter 238 may be rotatable relative to the end wall 122. The first counter 238 may include an axle 239. The axle 239 may be coupled to the end wall 122, permitting the first counter 238 to rotate on the axle 239 relative to the end wall 122. In some embodiments, the first counter 238 may have a display 237. The display 237 may include one or more components to display information. For example, the display 237 may be a dial having numbers from 1 to 12 circumferentially spaced about a face of the display 237.

The indicator system 234 may include at least one window 248. The first counter 238 can be coupled to the end wall so that a portion of the first counter 238 is visible through the at least one window 248. In some embodiments, the first counter 238 can be coupled to the end wall 122 so that the display 237 is visible through the at least one window 248. During assembly, the first counter 238 can be activated. For example, the motor or mainspring can be wound, initiating a count up or a countdown sequence. The tab 246 can be inserted into the first counter 238. In some embodiments, the tab 246 can interrupt motion of the clockwork. For example, the tab 246 may prevent the rotation of one or more gears in the clockwork. By interrupting the operation of the clockwork, the tab 246 can keep the motor energized during shipping or storage. During use, the first housing 118 is turned relative to the second housing 120. In response, the first counter 238 can be moved relative to the tab 246 and the ring 236. The relative motion can break the tab 246, allowing the first counter 238 to being counting up or down. The display 237 can display the relative time through the at least one window 248. In other embodiments, additional counters 238 can be used and displayed through additional windows 248 to track other aspects of therapy, for example, time since last dressing change. In some embodiments, the first counter 238 may count down. For example, the first counter 238 may be set during the manufacturing process to count down from a maximum of 12 days. At the start of therapy, the display 237 may show the number 12 through the window 248. After the passage of each twenty-four hour period, the display 237 may sequentially decrease the number visible through the window 248. In other embodiments, the first counter 238 may sequentially count up from zero, increasing the number at the desired interval.

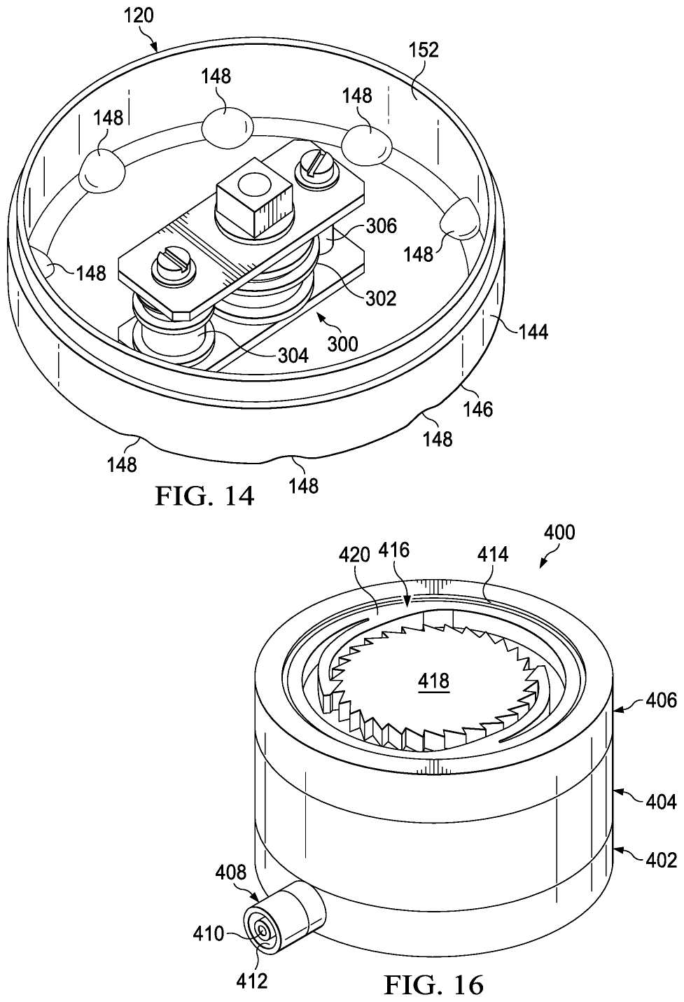

FIG. 14 is a perspective view illustrating additional details of another embodiment of the negative-pressure source 102. The second housing 120 may have a clockwork 300 disposed in the cavity 152. The second housing 120 can include the negative-pressure reservoir 154, the biasing member 156, and the regulator 158, none of which are shown in FIG. 14 for ease of illustration. The clockwork 300 can be a mechanical mechanism having a motor. The motor can include a mainspring capable of storing energy. Often, energy can be stored in the mainspring by winding the motor. The motor can then drive a plurality of gears. The clockwork 300 may include a balance wheel and a train gear or a fan fly to parse out regular intervals of time. The clockwork 300 may be capable of marking increasing or decreasing intervals of time. In some embodiments, the biasing member 156 can be coupled to the clockwork 300 and be adapted to function as the motor. The clockwork 300 can include a main gear 302 that can be coupled to the biasing member 156. The clockwork 300 can also include one or more ancillary gears, for example, the clockwork mechanism includes a first ancillary gear 304 and a second ancillary gear 306. The first ancillary gear 304 and the second ancillary gear 306 can be driven by the main gear 302. In some embodiments, the main gear 302 can drive the first ancillary gear 304 and the second ancillary gear 306 at the same rate. In other embodiments, the main gear 302 can drive the first ancillary gear 304 and the second ancillary gear 306 at different rates.

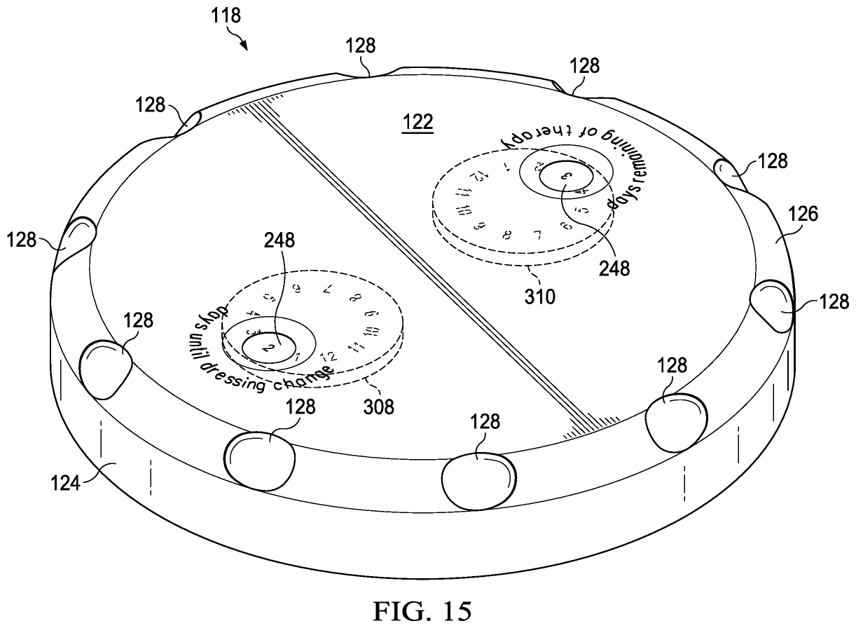

FIG. 15 is a perspective view illustrating additional details of another embodiment of the negative-pressure source 102. The first housing 118 may have the windows 248. A dressing gear 308 and a therapy gear 310 can be coupled to the first housing 118. The dressing gear 308 and the therapy gear 310 can be another type of charge status indicator or life status indicator. In some embodiments, the dressing gear 308 and the therapy gear 310 can be positioned so that at least a portion of the dressing gear 308 and a portion of the therapy gear 310 can be seen through the windows 248, respectively. The dressing gear 308 and the therapy gear 310 can be operatively coupled to one or more of the first ancillary gear 304 and the second ancillary gear 306. Motion of the first ancillary gear 304 and the second ancillary gear 306 can cause motion of the dressing gear 308 and the therapy gear 310. As the main gear 302 is driven by the biasing member 156 through rotation of the first housing 118 relative to the second housing 120, the main gear 302 can drive the first ancillary gear 304 and the second ancillary gear 306 to display relevant information through the windows using the dressing gear 308 and the therapy gear 310.

In operation, the first housing 118 can be rotated relative to the second housing 120, moving the biasing member 156 from the first position of FIG. 3 to the second position of FIG. 4. As the biasing member 156 draws the second end 172 of the biasing member 156 to the first position of FIG. 3, the biasing member 156 can drive the main gear 302. In response, the first ancillary gear 304 and the second ancillary gear 306, the dressing gear 308 and the therapy gear 310 can all be driven. The gear ratio between the main gear 302, the first ancillary gear 304, and the second ancillary gear 306; the gear ratio between the first ancillary gear 304 and the dressing gear 308; and the gear ratio between the second ancillary gear 306 and the therapy gear 310 can be selected to coincide with a status of therapy. For example, the gear ratios can be selected so that as the second end 172 of the biasing member 156 moves form the second position to the first position, the dressing gear 308 and the therapy gear 310 will indicate the number of days remaining until the dressing 104 should be changed and/or the negative-pressure source 102 is no longer providing negative-pressure therapy.

FIG. 16 is a perspective view illustrating additional details of another embodiment of the negative-pressure source 102 of FIG. 1, for example, a negative-pressure source 400. The negative-pressure source 400 can include a first housing 402, a second housing 404, and a third housing 406. The first housing 402 may be a disc shaped bodying having a conduit adapter 408 coupled to a side wall of the first housing 402. In some embodiments, the conduit adapter 408 may be a multi-lumen adapter. For example, the conduit adapter 408 may have a central lumen 410 and an annular lumen 412 surrounding the central lumen 410.

The second housing 404 may be a carriage and may be a disc shaped body. The second housing 404 may be disposed adjacent the first housing 402. In some embodiments, the second housing 404 may be coupled to the first housing 402.

The third housing 406 may be a disc-shaped body. The third housing 406 may be disposed adjacent to the second housing 404. In some embodiments, the third housing 406 may be coupled to the second housing 404. In some embodiments, the third housing 406 may be rotatable relative to the second housing 404. The third housing 406 may include a recess 414 having a lockout 416. The lockout 416 may include a gear 418 and a pawl 420.

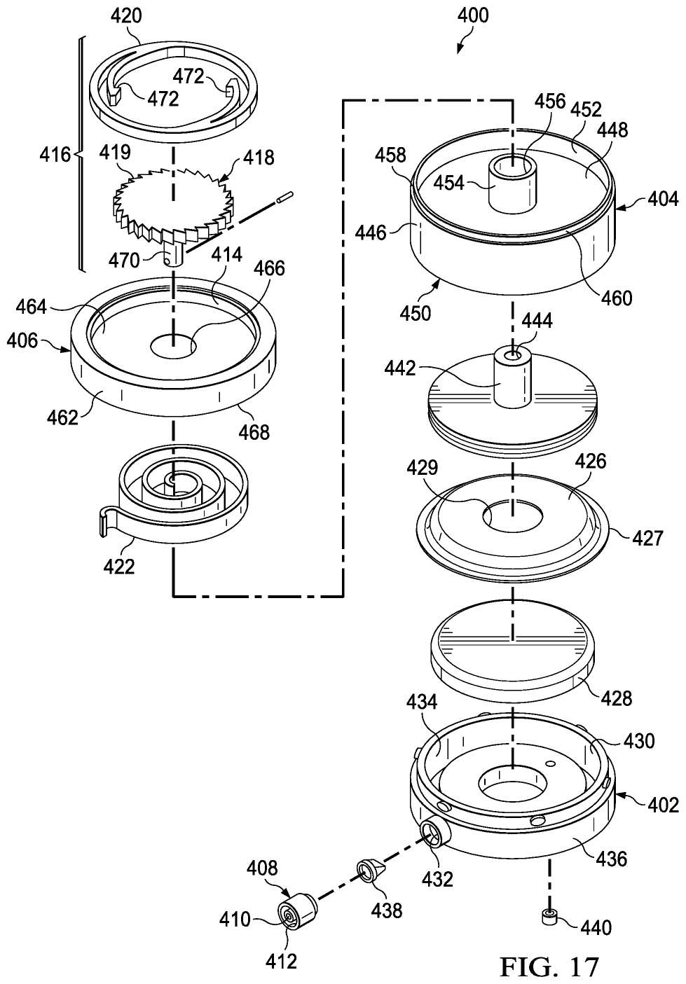

FIG. 17 is an assembly view illustrating additional details of the negative-pressure source 400 of FIG. 16. The negative-pressure source 400 can further include a main spring 422, a threaded body 424, a diaphragm 426, and a seal 428. The main spring 422 can be disposed between the third housing 406 and the second housing 404. The threaded body 424 can be disposed between the second housing 404 and the first housing 402. The diaphragm 426 can be disposed between the threaded body 424 and the first housing 402. The seal 428 can be disposed between the diaphragm 426 and the first housing 402.

The first housing 402 may have an end wall 434 and a side wall 436. The side wall 436 may be annular. The side wall 436 can be coupled to the end wall 434 to form a cavity 430. In some embodiments, the side wall 436 may be annular. A therapy port 432 can be coupled to the side wall 436. The therapy port 432 may be an opening or aperture formed in the side wall 436. The therapy port 432 can extend through the side wall 436. In some embodiments, the therapy port 432 can provide fluid communication with the cavity 430 across the side wall 436. A valve 438 can be disposed in the therapy port 432. In some embodiments, the valve 438 can be a one-way valve permitting fluid communication into the cavity 430 across the side wall 436 and preventing fluid communication out of the cavity 430 across the side wall 436. The valve 438 can include a check valve, a ball valve, a flap valve, an umbrella valve, or a duckbill valve. The conduit adapter 408 can be coupled to the therapy port 432. The first housing 402 can further include a vent valve 440. The vent valve 440 can be positioned in the end wall 434. In some embodiments, the vent valve 440 can be a one-way valve permitting fluid communication out of the cavity 430 across the end wall 434 and preventing fluid communication into the cavity 430 across the end wall 434. The vent valve 440 can include a check valve, a ball valve, a flap valve, an umbrella valve, or a duckbill valve.

In some embodiments, the diaphragm 426 can be disposed within the cavity 430 of the first housing 402. The diaphragm 426 may have a diaphragm radius and a peripheral edge 427 disposed at the end of the diaphragm radius. The peripheral edge 427 may contact and seal to an inner surface of the side wall 436 of the first housing 402 to form a pump chamber between the diaphragm 426 and the end wall 434. The diaphragm 426 may also include an aperture 429 extending through the diaphragm 426. The diaphragm 426 be a flexible member or membrane. In some embodiments, the diaphragm 426 may be formed from a rubber, Santoprene™, silicone, polyurethane, or other similarly flexible material. The seal 428 can be disposed in the cavity 430 of the first housing 402 between the end wall 434 and the diaphragm 426. The seal 428 may have a seal radius that is less than the diaphragm radius. In some embodiments, the seal 428 can be disposed over the aperture 429, preventing fluid communication across the diaphragm 426 through the aperture 429. The seal 428 can be formed from a rubber, Santoprene™, silicone, polyurethane, or other similarly flexible material. In some embodiments, an absorbent, desiccant, or other fluid solidifier can be disposed in the cavity 430.

The threaded body 424 can be coupled to the diaphragm 426. The threaded body 424 may be a disc shaped body having a threaded body radius that is less than the diaphragm radius. In some embodiments, the threaded body 424 may not contact the side wall 436 of the first housing 402. The threaded body 424 may have a projection 442 coupled to a side of the threaded body 424 opposite the diaphragm 426. The projection 442 may be a cylindrical body having a first end coupled to the threaded body 424, a second end spaced apart from the threaded body 424, and a sidewall coupling the first end to the second end. The projection 460 may have a radius that is less than the threaded body 424 radius. The projection 442 may have a bore 444 extending through the projection 442 from the second end to the first end. In some embodiments, the bore 444 be open at both the first end and the second end. The bore 444 may be threaded from the first end to the second end. In some embodiments, the thread can be an open thread capable of being threaded through 360 degrees. The threaded body 424 may be rotationally fixed to the side wall 436 of the cavity 430 of the first housing 402. For example, the threaded body 424 may include one or more detents configured to mate with a corresponding slot formed in the side wall 436 of the cavity 430 of the first housing 402. The detents may prevent relative rotation between the threaded body 424 and the first housing 402. The detents may permit axial translation between the threaded body 424 and the first housing 402.

The second housing 404 may include a side wall 446. The side wall 446 may be an annular wall having a first surface or exterior surface and a second surface or interior surface extending between a first end adjacent to the first housing 402 and a second end opposite the first end. A projection 460 may extend from the second end of the side wall 446. The projection 460 may be annular having an inner radius equal to an inner radius of the side wall 446 and an outer radius less than an outer radius of the side wall 446. In some embodiments, the projection 460 may form a shoulder 458 between the exterior surface of the side wall 446 and the projection 460. The shoulder 458 may have a radial width of about one-half a thickness of the side wall 446. The second housing 404 can also include an end wall 448 disposed between the first end and the second end of the side wall 446 and coupled to the interior surface of the side wall 446. In some embodiments, the end wall 448 can be coupled about halfway between the first end and the second end of the side wall 446, forming a first cavity 450 proximate to the first housing 402 and a second cavity 452 proximate to the third housing 406. An axle 454 may be coupled to the end wall 448. In some embodiments, the axle 454 may be a cylindrical body having a bore 456 extending through the axle 454. The axle 454 may be coupled to the end wall 448 and extend through the second cavity 452. The bore 456 may have a radius that is less than the radius of the projection 442, permitting the projection 442 to be inserted into the bore 456.

The main spring 422 can be disposed in the second cavity 452 and coupled to the axle 454 and the third housing 406. The main spring 422 can be a type of motor, for example, a torsion metal ribbon spring. In some embodiments, the main spring 422 can be formed from spring steel and be considered a type of constant-force spring. The main spring 422 can be a hardened and blued steel or a steel alloy. An inner end of the main spring 422 can be coupled to one of the second housing 404 and the third housing 406 and an outer end of the main spring 422 can be coupled to the other of the third housing 406 and the second housing 404. In some embodiments, the third housing 406 is rotatable relative to the second housing 404. The main spring 422, having separate ends coupled to the second housing 404 and the third housing 406, can be wound or energized in response to rotation of the third housing 406 relative to the second housing 404.

The third housing 406 may have a side wall 462. The side wall 462 may be an annular wall having a first end proximate to the second housing 404, a second end opposite the first end, a first surface or interior surface, and a second surface or exterior surface, both extending from the first end to the second end. An end wall 464 may be coupled to the first end of the side wall 462, forming the recess 414. The third housing 406 may also include an aperture 466 formed in the end wall 464. The aperture 466 may have a diameter greater than the diameter of the axle 454 of the second housing 404, permitting the axle 454 to be inserted into the aperture 466.

The gear 418 may have a plurality of teeth 419 and an axle 470. The axle 470 may be coupled to the gear 418 and be insertable through the aperture 466, thorough the bore 456 and into the bore 444. In some embodiments, the axle 470 may be a screw having a thread extending the length of the axle 470. The thread of the axle 470 may be configured to mate with the corresponding thread of the bore 444. The pawl 420 can be coupled to the third housing 406 and have a hammer 472 that engages the plurality of teeth 419 to limit rotation of the third housing 406 relative to the gear 418 to a first rotational direction.

In operation, the third housing 406 can be rotated relative to the second housing 404, energizing the main spring 422. In some embodiments, the hammers 472 can engage the teeth 419 of the gear 418, causing the gear 418 to rotate with the third housing 406 relative to the second housing 404. The relative rotation between the third housing 406 and the gear 418, being coupled to the axle 470, and the second housing 404 can also drive the threaded body 424 axially toward the first housing 402. In response, the diaphragm 426 can be driven downward, evacuating fluid from the cavity 430 through the vent valve 440. The main spring 422, energized by the relative rotation between the third housing 406 and the second housing 404 can urge the third housing 406 to rotate in the opposite direction. The hammers 472 can allow the teeth 419 to slide past the hammer as the third housing 406 rotates relative to the second housing 404. The reverse rotation can drive the threaded body 424 in the opposite direction, moving the diaphragm 426 axially out of the cavity 430. As the volume of the cavity 430 increases with the movement of the diaphragm 426, fluid can be drawn into the cavity 430 through the therapy port 432.

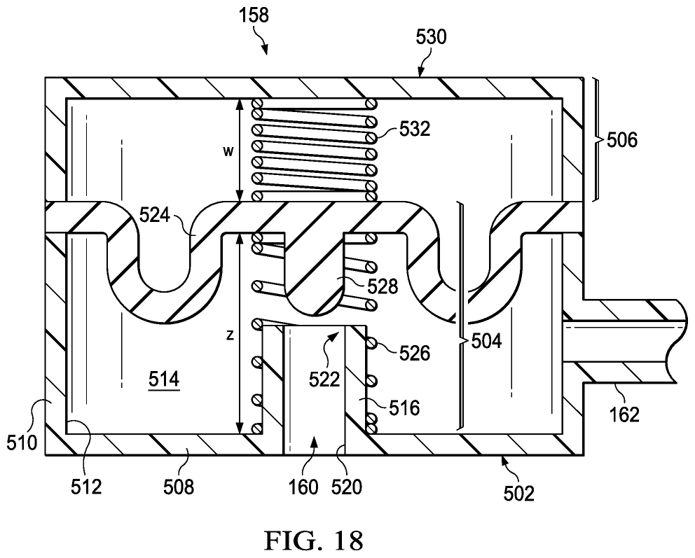

FIG. 18 is a section view illustrating additional details of the regulator 158. The regulator 158 may be a type of controller 112 and can be used with each of the negative-pressure sources described herein. The regulator 158 may include a housing 502, a regulator valve 504, and a valve calibrator 506. The housing 502 may have an end wall 508, one or more side walls 510, and an open end 512 opposite the end wall 508. The side walls 510 may be coupled to peripheral portions of and generally perpendicular to the end wall 508.

The housing 502 may form a charging chamber 514. In some embodiments, the charging chamber 514 may be disposed between the end wall 508 and the side walls 510. The housing 502 may be formed of a material having a sufficient strength to resist collapse if a reduced pressure is supplied to the charging chamber 514, such as metals, hard plastics, or other suitable materials. For example, the housing 502 may resist collapse if a reduced pressure of about 150 mm Hg (−150 mm Hg gauge pressure) is supplied to the charging chamber 514. In other exemplary embodiments, the housing 502 may resist collapse if a reduced pressure of about 600 mm Hg (−600 mm Hg gauge pressure) is supplied to the charging chamber 514.

The charging chamber 514 may include the suction port 162 and the therapy port 160. The suction port 162 may be disposed in one of the side walls 510 of the charging chamber 514 and may be fluidly coupled to the charging chamber 514. In some embodiments, the suction port 162 may be configured to be fluidly coupled to a supply of reduced pressure, such as an electric pump, a manual pump, or wall-suction source, for example. In some embodiments, the suction port 162 may be fluidly coupled to the negative-pressure reservoir 154. A one-way valve may be disposed in the suction port 162 and oriented to prevent fluid flow into the charging chamber 514 through the suction port 162 and permit fluid flow out of the charging chamber 514 through the suction port 162.

In some embodiments, the therapy port 160 may be disposed in the end wall 508, as shown in the illustrative embodiment of FIG. 18. The therapy port 160 may fluidly couple the charging chamber 514 to another device, such as the tube 164 of the dressing 104. In some embodiments, the therapy port 160 may have a cylindrical wall 516 and a central passage 520 that extends from the end wall 508 into the charging chamber 514. The cylindrical wall 516 may include a portion extending into the charging chamber 514 so that the therapy port 160 terminates near a center portion of the open end 512. In some embodiments, the therapy port 160 may be disposed in other locations of the end wall 508. In some embodiments, the therapy port 160 may have a valve seat 522 on the distal end. The valve seat 522 may provide a tapered or beveled edge proximate to the central passage 520 of the therapy port 160.

In some embodiments, the open end 512 may provide a fluid path between the therapy port 160 and the charging chamber 514, which may be controlled by the regulator valve 504. The regulator valve 504 may include a valve member 524 and a regulator spring 526. The regulator valve 504 can be coupled to the open end 512 and operably associated with the therapy port 160 to regulate fluid communication between the charging chamber 514 and the supply chamber 1010. The regulator valve 504 can be biased to either open or close the therapy port 160. The regulator valve 504 may be coupled to ends of the side walls 510 of the housing 502, opposite the end wall 508 of the housing 502. In some embodiments, the regulator valve 504 may substantially limit or prevent fluid communication through the open end 512 of the housing 502.

In some embodiments, the valve member 524 may be a flexible membrane, such as a diaphragm. In some embodiments, the valve member 524 may have a generally disc-like shape with a diameter larger than the diameter of the open end 512. In other embodiments, the valve member 524 may have a shape matched to a shape of the open end 512, for example, square, rectangular, ovoid, triangular, or amorphous shapes. The valve member 524 may have peripheral portions coupled to the side walls 510, and the valve member 524 may extend across the open end 512. If the valve member 524 is coupled to the side walls 510, the valve member 524 may fluidly isolate the supply chamber 1010 from the ambient environment surrounding the regulator 158. For example, a difference in the pressures in the charging chamber 514 and the ambient environment may cause deflection of the valve member 524. In some embodiments, the valve member 524 may be formed from a silicone material. In some embodiments, the valve member 524 may have a hardness rating between about 100 Shore A and about 50 Shore A.

In some embodiments, the valve member 524 may include an enlarged portion 528 configured to engage the valve seat 522. The valve member 524 may be positioned so that the enlarged portion 528 of the valve member 524 may engage a beveled edge of the valve seat 522 of the therapy port 160 in a closed position. If engaged in such a manner, the valve member 524 can substantially prevent fluid communication through the central passage 520 of the therapy port 160.

The regulator spring 526 may be disposed on the therapy port 160 so that the regulator spring 526 circumscribes the therapy port 160. The regulator spring 526 may have a first end adjacent to the end wall 508. In some embodiments, the first end of the regulator spring 526 may contact the end wall 508 so that the regulator spring 526 may be compressed against the end wall 508. A second end of the regulator spring 526 may be proximate to the distal end of the therapy port 160. The regulator spring 526 may have a length Z if in a relaxed position. In the relaxed position, the regulator spring 526 may be longer than the therapy port 160.

The valve calibrator 506 may include a regulator cap 530 and a calibration spring 532. The regulator cap 530 may be coupled to the housing 502 so that the regulator cap 530 is adjacent to the open end 512. In some embodiments, the regulator cap 530 covers the open end 512 of the housing 502 and includes an opening permitting ambient pressure to be communicated to an area adjacent the valve member 524.

In some embodiments, the calibration spring 532 may be disposed between the valve member 524 and the regulator cap 530. In some embodiments, an end of the calibration spring 532 may contact the valve member 524 on an opposite side of the valve member 524 from the regulator spring 526. The calibration spring 532 may have a first end adjacent to the valve member 524. In some embodiments, the first end of the calibration spring 532 may contact the valve member 524 on an opposite side of the valve member 524 from the enlarged portion 528, allowing the calibration spring 532 to be compressed against the valve member 524 opposite the regulator spring 526. A second end of the calibration spring 532 may extend to and contact the regulator cap 530. The calibration spring 532 may have a length W if in a relaxed position.

In some embodiments, a differential force may operate on the valve member 524. The differential force may be a force generated by a difference in pressures between the charging chamber 514 and the ambient environment of the regulator 158. The pressure in the charging chamber 514 may also be referred to as a supply pressure or the manifold pressure. If the supply pressure in the charging chamber 514 and the pressure in the ambient environment are substantially equal, the differential force may be approximately zero. If the supply pressure in the charging chamber 514 is less than the ambient pressure, for example, if the regulator 158 is being used to provide reduced-pressure therapy, the differential force may act to urge the valve member 524 toward the distal end of the therapy port 160.

In some embodiments, the regulator spring 526 may exert a force in response to movement of the regulator spring 526 from the relaxed position. If the regulator spring 526 is disposed in the charging chamber 514, the regulator spring 526 may be moved from the relaxed position so that the regulator spring 526 has a length Z₁. If the regulator spring 526 is compressed to the length Z₁, the regulator spring 526 may exert a regulator force urging the valve member 524 away from the valve seat 522 of the therapy port 160. Generally, the regulator force exerted on the valve member 524 may be proportional to a distance the regulator spring is moved from the relaxed position. Generally, the regulator spring 526 may be selected so that the differential force may overcome the regulator force if the supply pressure is about the therapy pressure. If the differential force overcomes the regulator force, the valve member 524 may contact the therapy port 160 and prevent fluid communication through the therapy port 160.

In some embodiments, the calibration spring 532 may exert a force in response to movement of the calibration spring 532 from the relaxed position. If the calibration spring 532 is disposed in the calibrator mount 1052, the calibration spring 532 may be moved from the relaxed position so that the calibration spring 532 may have a length W₁. If the calibration spring 532 is compressed to the length W₁, the calibration spring 532 may exert a calibration force urging the valve member 524 toward the valve seat 522 of the therapy port 160. Generally, the force exerted on the valve member 524 may be proportional to a distance the calibration spring 532 is moved from the relaxed position. In some embodiments, the force of the regulator spring 526 and the force of the calibration spring 532 may urge the valve member 524 in opposite directions.

In some embodiments, the calibration spring 532 may exert a force that assists the differential force in urging the valve member 524 into contact with the valve seat 522 of the therapy port 160. The force exerted by the calibration spring 532 may be used to calibrate the regulator 158 to the desired therapy pressure. For example, the regulator 158 may be tested to determine if the regulator 158 supplies reduced pressure at a desired therapy pressure with no calibration force. If the regulator 158 fails to provide the therapy pressure, the calibration spring 532 may be used to increase the calibration force applied by the calibration spring 532. In other embodiments, if the regulator 158 has already been calibrated, the regulator 158 may be tested to determine if the regulator 158 supplies reduced pressure at a desired therapy pressure. If the regulator 158 provides insufficient reduced pressure, the calibration spring 532 may be used to decrease the calibration force applied by the calibration spring 532, thereby increasing the required differential force to overcome the regulator force.

If the differential force plus the calibration force is greater than the force of the regulator spring 526 acting on the valve member 524, the valve member 524 may be urged into contact with the distal end of the therapy port 160 to prevent fluid communication through the therapy port 160 in a closed position. In response, the regulator spring 526 may be compressed to a length Z2. If the differential force plus the calibration force is less than the force of the regulator spring 526, the valve member 524 may be urged away from the distal end of the therapy port 160 to permit fluid communication through the therapy port 160. The calibration spring 532 may be used to control the differential force required to overcome the regulator spring 526. For example, if less reduced pressure is required, the calibration spring 532 may be selected so that the calibration spring 532 has a length W2. Thus, the displacement of the calibration spring 532 can be controlled to calibrate the differential pressure, so that the force of the regulator spring 526 may be overcome if the therapy pressure is reached in the charging chamber 514.

The systems, apparatuses, and methods described herein may provide significant advantages. For example, the bellows or foam structure can be adapted to provide a linear force and thus linear vacuum generation. The negative-pressure source can be larger, flat, and have a rotation form-factor that may be easier to operate for users with limited dexterity. The negative-pressure source can include indicators for life and charge status that are more instructive than simply charged or not charged and can provide indications of the duration of therapy that the user can expect from the system. Some embodiments use a simple and low cost digital indicator that can provide information to the user about the status of the negative-pressure source and the tissue site. The data collected by the digital indicator may be extracted via a range of means. A simple chart on a small LCD display of the digital indicator may assist with motivating the patient to use the device and provide confidence to the user that the negative-pressure therapy is being provided. The indicators described herein can also log how often the negative-pressure source needs to be recharged and can provide the flow/leak rate within the system/dressing. Some embodiments of the negative-pressure source include reusable or changeable bellows.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A negative-pressure source comprising:
a first housing;
a second housing configured to be coupled to the first housing to form a cavity, the first housing and the second housing rotatable relative to each other on a common axis;
a membrane disposed in the cavity and sealed to the second housing to form a first chamber and a second chamber;
a motor disposed in the first chamber;
a clockwork coupled to the motor and configured to be driven by the motor; and
a threaded body disposed in the first chamber and configured to be driven by the motor, the threaded body configured to rotate on the common axis, the threaded body further configured to displace the membrane in response to rotation on the common axis.

2. The negative-pressure source of claim 1, wherein rotation of the first housing relative to the second housing energizes the motor.

3. The negative-pressure source of claim 1, wherein the rotation of the threaded body is configured to displace the membrane axially, generating a negative pressure in the second chamber.

4. The negative-pressure source of claim 1, wherein the clockwork is configured to drive a charge status indicator.

5. The negative-pressure source of claim 1, wherein the clockwork is configured to drive a life status indicator.

6. A negative-pressure source comprising:
a bottom housing having a cylindrical shape, a first end, and a second end, and a cavity depending from the first end toward the second end;
a diaphragm disposed in the cavity and having a peripheral portion configured to seal to the bottom housing to form a pump chamber;
a threaded body having a first end and a second end, the second end coupled to the diaphragm;
a carriage coupled to the first end of the threaded body;
a motor disposed in the carriage;
a top housing coupled to the motor and configured to rotate relative to the bottom housing to energize the motor; and
a clockwork coupled to the first end of the threaded body, the clockwork configured to be driven by the motor.

7. The negative-pressure source of claim 6, further comprising an absorbent disposed in the pump chamber.

8. The negative-pressure source of claim 6, further comprising:
an inlet coupled to the bottom housing and fluidly coupled to the pump chamber; and
a one-way valve coupled to the inlet, the one-way valve configured to permit fluid communication into the pump chamber and prevent fluid communication out of the pump chamber.

9. The negative-pressure source of claim 6, further comprising a seal disposed in the pump chamber and coupled to the diaphragm.

10. The negative-pressure source of claim 9, further comprising a seal configured to be coupled to the second end of the threaded body and coupled to an opposite side of the diaphragm from the threaded body.

11. The negative-pressure source of claim 6, wherein the threaded body has a peripheral portion configured to engage a wall of the bottom housing to limit rotation of the threaded body relative to the bottom housing.

12. The negative-pressure source of claim 6, wherein the carriage comprises:
a cylindrical body having a first end and a second end;
a carriage cavity depending into the cylindrical body from the first end toward the second end; and
the threaded body being coupled to the carriage opposite the carriage cavity.

13. The negative-pressure source of claim 6, further comprising a lockout coupled to the top housing and configured to permit rotation of the top housing relative to the bottom housing in a single direction.

14. The negative-pressure source of claim 6, wherein the clockwork is configured to drive a charge status indicator.

15. The negative-pressure source of claim 6, wherein the clockwork is configured to drive a life status indicator.

16. A method for providing negative-pressure therapy, the method comprising:
disposing a dressing adjacent a tissue site;
providing a negative-pressure source comprising:
a bottom housing having a cylindrical shape, a first end, and a second end, and a cavity depending from the first end toward the second end;
a diaphragm disposed in the cavity and having a peripheral portion configured to seal to the bottom housing to form a pump chamber;
a threaded body having a first end and a second end, the second end coupled to the diaphragm;
a carriage coupled to the first end of the threaded body;
a motor disposed in the carriage;
a top housing coupled to the motor and configured to rotate relative to the bottom housing to energize the motor; and
a clockwork coupled to the first end of the threaded body, the clockwork configured to be driven by the motor;
fluidly coupling the negative-pressure source to the dressing;
rotating the top housing relative to the bottom housing to displace the diaphragm; and
drawing fluid from the dressing into the pump chamber.

17. The method of claim 16, wherein the rotation of the top housing to the bottom housing drives a charge status indicator.

18. The method of claim 16, wherein the rotation of the top housing to the bottom housing drives a life status indicator.

19. The method of claim 16, wherein rotating the top housing relative to the bottom housing comprises:
energizing the motor;
rotating the carriage; and
rotating the threaded body.

20. The method of claim 19, wherein rotating the threaded body comprises moving the threaded body axially, drawing the diaphragm toward the carriage and expanding the pump chamber.

21. The systems, apparatuses, and methods substantially as described herein.
